(19) United States
(12) Patent Application Publication
(10) Pub. No.: US 2016/0216271 A1
(43) Pub. Date: Jul. 28, 2016

(54) Fluorescent Polymer Dots, Methods For Their Preparation, And Uses Thereof

(71) Applicants: AGENCY FOR SCIENCE, TECHNOLOGY AND RESEARCH, Connexis, Singapore (SG); NATIONAL UNIVERSITY OF SINGAPORE, Singapore (SG)

(72) Inventors: Kai Li, Singapore (SG); Bin Liu, Singapore (SG); Guangxue Feng, Singapore (SG); Jie Liu, Singapore (SG)

(73) Assignees: AGENCY FOR SCIENCE, TECHNOLOGY AND RESEARCH, Connexis, Singapore (SG); NATIONAL UNIVERSITY OF SINGAPORE, Singapore (SG)

(21) Appl. No.: 14/916,172

(22) PCT Filed: Sep. 2, 2014
(86) PCT No.: PCT/SG2014/000416

§ 371 Date: Mar. 2, 2016

(30) Foreign Application Priority Data
Sep. 2, 2013 (SG) 2013066022

(51) Int. Cl. G01N 33/58; C09K 11/06; G01N 21/64; C09K 11/02

(52) U.S. Cl. CPC G01N 33/582; C09K 11/025; C09K 11/06; G01N 21/6428; G01N 33/587; C09K 2211/1475; Y10S 977/774; Y10S 977/892; Y10S 977/904; B82Y 40/00

(57) ABSTRACT

There is provided a fluorescent polymer dot (FPdot) comprising one or more fluorescent conjugated polymers forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties. There is also provided a method for preparing the fluorescent polymer dot comprising preparing a mixture of conjugated polymer and amphiphilic molecule in an aprotic solvent; and adding said mixture to a protic solvent. The fluorescent polymer dots may be useful in biomedical imaging applications.

### TECHNICAL FIELD

The present invention generally relates to fluorescent polymer dots (FPdots), methods for their preparation, and uses thereof. The fluorescent polymer dots may be useful in biomedical imaging applications.

### BACKGROUND

Fluorescent probes play a vital role in fundamental biological and biomedical imaging applications. Among conventional fluorescent probes, inorganic quantum dots have attracted great interest. However, quantum dots suffer from potential degradation caused by reactive oxygen species during imaging, further resulting in fluorescence decrease and release of toxic heavy metal ions. Conjugated polymers with n-electron delocalized backbones have been considered as promising in designing fluorescent nanoparticles for bioimaging. Although conjugated polymer-based nanoparticles with comparable sizes to quantum dots have been developed, their applications have been limited due to the limited types of biomolecules they are able to conjugate to. Conventionally conjugated polymer-based nanoparticles further display low fluorescence quantum yield due to fluorescence quenching. Conventional conjugated polymer-based nanoparticles further suffer from problems related to photostability, low cell-tracing life and may display low biocompatibility.

There is a therefore a need to provide fluorescent nanoparticles and methods for their preparation that overcome, or at least ameliorate, one or more of the disadvantages described above.

### SUMMARY

According to a first aspect, there is provided a fluorescent polymer dot comprising one or more fluorescent conjugated polymers forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties.

Advantageously, the disclosed fluorescent polymer dots may display superior biocompatibility.

Advantageously, the disclosed fluorescent polymer dots may be of uniform size and low cytotoxicity which makes them useful for bioimaging purposes. As the disclosed fluorescent polymer dots may further be of comparable size, similar or superior photostability and similar or superior labelling efficiency when compared with inorganic quantum dots, the disclosed fluorescent polymer dots may overcome the abovementioned limitations of quantum dots and conventional conjugated polymer-based nanoparticles in bioimaging applications and may be consequently be superior alternatives in bioimaging applications.

Further advantageously, the disclosed fluorescent polymer dots may display superior fluorescence quantum yield when compared to conventional, conjugated polymer-based nanoparticles which are known to experience fluorescence quenching.

Further advantageously, the size of the disclosed fluorescent polymer dots may be customizable. As the electronic characteristics of fluorescent polymer-dots may be affected by its size, the disclosed fluorescent polymer dots may be customized to provide a range of electronic characteristics that suit different needs.

The size of the disclosed fluorescent polymer dots advantageously may not change after time, e.g. after storage, thereby displaying good stability.

In one embodiment, the disclosed fluorescent polymer dots may be functionalized.

Advantageously, the attached functional group(s) may be customizable which allows for a wide variety of biomolecules to be conjugated to said fluorescent polymer dots allowing for a greater variety of bioimaging applications.

Further advantageously, the disclosed fluorescent polymer dots exhibit similar or superior performance in molar extinction coefficient, fluorescence quantum yield and long-term stability when compared with quantum dots and conventional conjugated polymer-based nanoparticles which is of importance in long-term cell tracing applications.

In another aspect, there is provided a method for preparing a fluorescent polymer dot, the method comprising the steps of:

a) preparing a mixture of fluorescent conjugated polymer and amphiphilic molecule in an aprotic solvent; and

b) adding said mixture to a protic solvent, to form the polymer dot comprising one or more fluorescent conjugated polymers forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties.

Advantageously, it may be able to control the size of the prepared fluorescent polymer dots by the disclosed methods. As the electronic characteristics of fluorescent polymer dots may be affected by its size, the disclosed fluorescent polymer dots may be customized to provide a range of electronic characteristics that suit different needs.

Further advantageously, the disclosed methods may provide fluorescent polymer dots with high quantum yield.

Also advantageously, the disclosed methods may be able to provide fluorescent polymer dots with customizable surface functional groups, thereby allowing for a wide variety of biomolecules to be conjugated to said fluorescent polymer dots allowing for a greater variety of bioimaging applications.

In another aspect, there is provided a method of detecting a target molecule in a biological sample, the method comprising contacting a biological sample with a disclosed fluorescent polymer dot.

Advantageously, the disclosed fluorescent polymer dots may display superior living cell tracing ability up to about 12 generations.

### DEFINITIONS

The following words and terms used herein shall have the meaning as indicated.

The term “aprotic solvent” as used herein refers to a polar solvent which does not contain acidic hydrogen and does not act as a hydrogen bond donor.

The term “protic solvent” as used herein refers to a solvent that contains a dissociable H⁺ ion.

The terms “fluorescent polymer dot” or “FPdot” as used herein refers to a polymer dot that displays fluorescent properties and comprises one or more fluorescent conjugated polymers and one or more amphiphilic molecules.

The term “nanoparticle” as used herein refers to particles possessing dimensions less than about 1000 nm.

The term “fluorescent conjugated polymer” as used herein refers to a polymer that displays fluorescent properties, including oligomers such as dimers, trimers etc. and copolymers, which are fully conjugated (i.e. are conjugated along the entire length of the polymer chain) or are partially conjugated (i.e. which include non-conjugated segments in addition to conjugated segments). The conjugated polymers may have II-electron delocalized backbones.

The term “amphiphilic molecule” or “amphiphilic matrix” as used herein refers to a molecule comprising both hydrophobic and hydrophilic segments within said molecule. The term “hydrophilic” in this context refers to the segment of the amphiphilic molecule having a high affinity for aqueous solutions, such as water. The term “hydrophobic” in this context refers to the segment of the amphiphilic molecule having a repulsion towards aqueous solutions, such as water.

The term “aliphatic” as used herein refers to an organic compound or radical characterized by a straight chain or branched chain structure, or closed ring structure, any of which may contains saturated carbon bonds, and optionally, one or more unconjugated carbon-carbon unsaturated bonds, such as a carbon-carbon double bond. For the purposes of this invention, the term “aliphatic” also includes “alicyclic” compounds defined hereinafter. The aliphatic groups may have from 1 to 24 carbon atoms, eg, 1, 2, 3, 4, 5, 6, 7, 8, 9, 10, 11, 12, 13, 14, 15, 16, 17, 18, 19, 20, 21, 22, 23, 24 carbon atoms.

The term “alkyl” as used herein refers to monovalent (“alkyl”) and divalent (“alkylene”) straight chain or branched chain saturated aliphatic groups having from 1 to 24 carbon atoms, eg, 1, 2, 3, 4, 5, 6, 7, 8, 9, 10, 11, 12, 13, 14, 15, 16, 17, 18, 19, 20, 21, 22, 23, or 24 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, undecyl, dodecyl and the like. Alkyl groups may be optionally substituted.

The term “alkoxy” or variants such as “alkoxide” as used herein refers to an —O-alkyl radical. Representative examples include, for example, methoxy, ethoxy, n-propoxy, isopropoxy, tert-butoxy, and the like.

The term “amino” includes an amine group (i.e., —NH₂) or a substituted amine group.

The term “carboxy” refers to the group —C(O)OH.

The term “imide” refers to the reaction product of a dicarboxylic acid, carboxylate, anhydride, of a dicarboxylic acid, or ester of a dicarboxylic acid and a polyamine, such as a compound comprising two acyl groups bound to nitrogen.

The term “lipid moiety” refers to a moiety comprising at least one lipid. The term “lipid” as used herein refers to small molecules having hydrophobic or amphiphilic properties and include, but are not limited to, fats, waxes, fatty acids, cholesterol, phospholipids, monoglycerides, diglycerides and triglycerides. The fatty acids can be saturated, mono-unsaturated or poly-unsaturated. Examples of fatty acids include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). The lipid moiety can include several fatty acid groups using branching groups such as lysine and other branched amines.

The term “functional group” as used herein refers to a chemical moiety that is capable of reacting with a recited group to form an ionic bond, a covalent bond, or combination thereof.

The term “biological molecule” as used herein refers to a peptide, protein, saccharide, polysaccharide, nucleotide, antibody, aptamer, or any other compound that can target a specific type of cell, such as cancer cells or other disease-type cells.

The term “core” as used with reference to the fluorescent polymer dot refers to the center portion of the fluorescent polymer dot that comprises the hydrophobic segments of said fluorescent polymer dot.

The term “shell” as used with reference to the fluorescent polymer dot refers to the outermost layer of the fluorescent polymer dot that comprises the hydrophilic segments of said fluorescent polymer dot.

The core and the shell of the fluorescent polymer dot may form a “core-shell” structure where hydrophilic segments provide the protective shell layer and prevent the hydrophobic core from a surrounding aqueous solution.

The term “heteroaryl” as used herein refers to an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. Heteroaryl groups may be optionally substituted.

The term “O-containing heteroaryl” as used herein refers to heteroaryl groups containing at least one oxygen ring atom. Non-limiting examples of suitable O-containing heteroaryls include diazole, benzodiazole, oxazole, benzooxazole, pyran, furan, and benzofuran. O-containing heteroaryl groups may be optionally substituted.

The term “cycloalkyl” as used herein refers to a non-aromatic mono- or multicyclic ring system comprising about 3 to about 10 carbon atoms. The cycloalkyl can be optionally substituted with one or more “ring system substituents” which may be the same or different, and are as defined herein. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

The term “cycloalkenyl” as used herein refers to a non-aromatic mono or multicyclic ring system comprising about 3 to about 10 carbon atoms which contains at least one carbon-carbon double bond. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl, as well as unsaturated moieties of the examples shown above for cycloalkyl. Cycloalkenyl groups may be optionally substituted.

The term “heterocycle” as used herein refers to a group comprising a covalently closed ring herein at least one atom forming the ring is a carbon atom and at least one atom forming the ring is a heteroatom. Heterocyclic rings may be formed by three, four, five, six, seven, eight, nine, or more than nine atoms, any of which may be saturated, partially unsaturated, or aromatic. Any number of those atoms may be heteroatoms (i.e., a heterocyclic ring may comprise one, two, three, four, five, six, seven, eight, nine, or more than nine heteroatoms). Herein, whenever the number of carbon atoms in a heterocycle is indicated (e.g., C1-C6 heterocycle), at least one other atom (the heteroatom) must be present in the ring.

Designations such as “C1-C6 heterocycle” refer only to the number of carbon atoms in the ring and do not refer to the total number of atoms in the ring. It is understood that the heterocylic ring will have additional heteroatoms in the ring. In heterocycles comprising two or more heteroatoms, those two or more heteroatoms may be the same or different from one another. Heterocycles may be optionally substituted. Binding to a heterocycle can be at a heteroatom or via a carbon atom. Examples of heterocycles include heterocycloalkyls (where the ring contains fully saturated bonds) and heterocycloalkenyls (where the ring contains one or more unsaturated bonds).

The term “cyclic group” as used herein refers to an aryl, heteroaryl, cycloalkyl, cycloalkenyl or heterocycle as defined above. Cyclic groups may be optionally substituted. Cyclic groups may be monocyclic or polycyclic.

The term “optionally substituted” as used herein means the group to which this term refers may be unsubstituted, or may be substituted with one or more groups other than hydrogen provided that the indicated atom's normal valency is not exceeded, and that the substitution results in a stable compound. Such groups may be, for example, halogen, hydroxy, oxo, cyano, nitro, alkyl, alkoxy, haloalkyl, haloalkoxy, aryl4alkoxy, alkylthio, hydroxyalkyl, alkoxyalkyl, cycloalkyl, cycloalkylalkoxy, alkanoyl, alkoxycarbonyl, alkylsulfonyl, alkylsulfonyloxy, alkylsulfonylalkyl, arylsulfonyl, arylsulfonyloxy, arylsulfonylalkyl, alkylsulfonamido, alkylamido, alkylsulfonamidoalkyl, alkylamidoalkyl, arylsulfonamido, arylcarboxamido, arylsulfonamidoalkyl, arylcarboxamidoalkyl, aroyl, aroyl-4-alkyl, arylalkanoyl, acyl, aryl, arylalkyl, alkylaminoalkyl, a group RₓR_{y}N—, RₓOCO(CH₂)ₘ, RₓCON(R_{y}) (CH₂)ₘ, RₓR_{y}NCO(CH₂)ₘ, RₓR_{y}NSO₂(CH₂)ₘ or RₓSO₂NR_{y}(CH₂)ₘ (where each of Rₓ and R_{y} is independently selected from hydrogen or alkyl, or where appropriate RₓR_{y} forms part of carbocylic or heterocyclic ring and m is 0, 1, 2, 3 or 4), a group RₓR_{y}N(CH₂)ₚ— or RₓR_{y}N(CH₂)ₚO— (wherein p is 1, 2, 3 or 4); wherein when the substituent is RₓR_{y}N(CH₂)ₚ— or RₓR_{y}N(CH₂)ₚO, Rₓ with at least one CH₂ of the (CH₂)ₚ portion of the group may also form a carbocyclyl or heterocyclyl group and R_{y} may be hydrogen, alkyl.

The word “substantially”, does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of the disclosed fluorescent polymer dots, uses and methods of synthesis will now be disclosed.

### Fluorescent Polymer Dots (FPdots)

According to a first aspect, there is provided a fluorescent polymer dot comprising one or more fluorescent conjugated polymers forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties.

The fluorescent polymer dot may be a fluorescent conjugated polymer dot.

The aliphatic chain moieties may be unsaturated or saturated. The aliphatic chain moiety may comprise 1 to 24 carbon atoms, or 1, 2, 3, 4, 5, 6, 7, 8, 9, 10, 11, 12, 13, 14, 15, 16, 17, 18, 19, 20, 21, 22, 23, or 24 carbon atoms. The aliphatic chain moieties may form part of a lipid moiety.

Advantageously, the lipid moiety may be biocompatible, thus making the disclosed FPdots safe for use in the human or animal body.

The lipid moiety may be a 1,2-distearoyl-sn-glycero-3-phosphoethanolamino (DSPE) moiety, a 1,2-Dimyristoyl-sn-glycero-3-phosphoethanolamine (DMPE) moiety, a 1,2-Dilauroyl-sn-glycero-3-phosphoethanolamine (DLPE) moiety, or a 1,2-dipalmitoyl-sn-glycero-3-phosphoethanolamine (DPPE) moiety.

The lipid moiety may be a 1,2-distearoyl-sn-glycero-3-phosphoethanolamino (DSPE) moiety which advantageously has been approved by the Food and Drug Administration (FDA). Therefore, the disclosed FPdots advantageously display low cytotoxicity when used in a human or animal body and may display better biocompatibility when compared to other polymeric matrices, such as synthetic polymers like polystyrene.

The hydrophilic end of the amphiphilic molecule may comprise hydrophilic polymers. The hydrophilic polymers may be selected from the group consisting of polyoxyalkylene, polyalkylene glycol, and polycarboxyalkylene. The amphiphilic molecule may be selected from the group consisting of polyethylene glycol (PEG), polypropylene glycol, polybutylene glycol, polycarboxymethylene, polycarboxyethylene (or polyacrylic acid (PAA)), polycarboxypropylene and polycarboxybutylene.

The molecular weight of PEG may be in the range of about 1800 to about 5000, or about 1800 to about 4800, or about 1800 to about 4600, or about 1800 to about 4400, or about 1800 to about 4200, or about 1800 to about 4000, or about 1800 to about 3800, or about 1800 to about 3600, or about 1800 to about 3400, or about 1800 to about 3200, or about 1800 to about 3000, or about 1800 to about 2800, or about 1800 to about 2600, or about 1800 to about 2400, or about 1800 to about 2200, or about 1800 to about 2000, or about 2000 to about 5000, or about 2200 to about 5000, or about 2400 to about 5000, or about 2600 to about 5000, or about 2800 to about 5000, or about 2800 to about 5000, or about 3000 to about 5000, or about 3200 to about 5000, or about 3400 to about 5000, or about 3600 to about 5000, or about 3800 to about 5000, or about 4000 to about 5000, or about 4200 to about 5000, or about 4400 to about 5000, or about 4600 to about 5000, or about 4800 to about 5000, or about 1800, about 2000, about 2200, about 2400, about 2600, about 2800, about 3000, about 3200, about 3400, about 3600, about 3800, about 4000, about 4200, about 4400, about 4600, about 4800, or about 5000.

The molecular weight of PAA may be in the range of about 2000 to about 5000, or about 2000 to about 4800, or about 2000 to about 4600, or about 2000 to about 4400, or about 2000 to about 4200, or about 2000 to about 4000, or about 2000 to about 3800, or about 2000 to about 3600, or about 2000 to about 3400, or about 2000 to about 3200, or about 2000 to about 3000, or about 2000 to about 2800, or about 2000 to about 2600, or about 2000 to about 2400, or about 2000 to about 2200, or about 2000 to about 5000, or about 2200 to about 5000, or about 2400 to about 5000, or about 2600 to about 5000, or about 2800 to about 5000, or about 2800 to about 5000, or about 3000 to about 5000, or about 3200 to about 5000, or about 3400 to about 5000, or about 3600 to about 5000, or about 3800 to about 5000, or about 4000 to about 5000, or about 4200 to about 5000, or about 4400 to about 5000, or about 4600 to about 5000, or about 4800 to about 5000, or about 2000, about 2200, about 2400, about 2600, about 2800, about 3000, about 3200, about 3400, about 3600, about 3800, about 4000, about 4200, about 4400, about 4600, about 4800, or about 5000.

Advantageously, the surface polymer segments may provide excellent colloidal stability in the disclosed FPdots which is critical in biological imaging applications.

The hydrophilic end of the amphiphilic molecule may be attached to one or more functional groups. The functional groups may be attached through chemical or physical means such as through covalent bonding or ionic bonding. The functional groups may be reactive functional groups which may be capable of being conjugated to biomolecules. The functional groups may be selected from the group consisting of alkoxy, amino, carboxy, imide, azide and biotin. The functional groups may be selected from the group consisting of methoxy, ethoxy, propoxy, isopropoxy, n-, i-, sec-, tert-butoxy, C₅-alkoxy, C₆-alkyoxy, primary amine, secondary amine, tertiary amine, quarternary amine, aromatic amine, mono-, di-, or tri-substituted amine, —C(O)OH, saturated and unsaturated imide. The functional groups may be selected from the groups consisting of methoxy, amine, —C(O)OH, maleimide, and biotin. The functional groups may be further optionally substituted.

Advantageously, the type of functional group to be attached may be customizable which allows for a wide variety of biomolecules to be conjugated to the disclosed FPdots allowing for a greater variety of bioimaging applications. The functional groups may be chosen based on the target biomolecule. For example, the functional group may be selected from groups that specifically react or interact with functionalities on the surface of a protein, such as hydroxyl, amine, and carboxylic acid groups, or that recognize specific surface areas, such as an antibody, a lectin or a receptor-specific ligand, or interacts with the active site of enzymes. Those skilled in the art can select from a library of functionalities to accomplish this interaction.

The amphiphilic molecule may be selected from the group consisting of 1,2-distearoyl-sn-glycero-3-phosphoethanolamino-N-[(polyethylene glycol)-2000]- (DSPE-PEG), 1,2-Dimyristoyl-sn-glycero-3-phosphoethanolamine N-[(polyethylene glycol)-2000]- (DMPE-PEG), 1,2-Dilauroyl-sn-glycero-3-phosphoethanolamine-N-[(polyethylene glycol)-2000]- (DLPE-PEG), 1,2-dipalmitoyl-sn-glycero-3-phosphoethanolamine-N-[(polyethylene glycol)-2000]- (DPPE-PEG), 1,2-distearoyl-sn-glycero-3-phosphoethanolamino-N-[amino(polyacrylic acid)]- (DSPE-PAA), 1,2-distearoyl-sn-glycero-3-phosphoethanolamino-N-[amino(polyacrylic acid)]- (DMPE-PAA), 1,2-distearoyl-sn-glycero-3-phosphoethanolamino-N-[amino(polyacrylic acid)]- (DLPE-PAA) and 1,2-distearoyl-sn-glycero-3-phosphoethanolamino-N-[amino(polyacrylic acid)]- (DPPE-PAA) derivatives. The amphiphilic molecule may be selected from the group consisting of:
1,2-distearoyl-sn-glycero-3-phosphoethanolamine-N-[(polyethylene glycol)-2000] (DSPE-PEG);
1,2-distearoyl-sn-glycero-3-phosphoethanolamine-N-[methoxy(polyethylene glycol)-2000] (DSPE-PEG-OCH₃),
1,2-Distearoyl-sn-glycero-3-phosphoethanolamine-N-[amino(polyethylene glycol)-2000] (DSPE-PEG-NH₂);
1,2-distearoyl-sn-glycero-3-phosphoethanolamine-N-[carboxy(polyethylene glycol)-2000] (DSPE-PEG-COOH);
1,2-distearoyl-sn-glycero-3-phosphoethanolamine-N-[maleimide(polyethylene glycol)-2000] (DSPE-PEG-maleimide);
1,2-distearoyl-sn-glycero-3-phosphoethanolamine-N-[biotinyl(polyethylene glycol)-2000] (DSPE-PEG-biotin);
1,2-Dimyristoyl-sn-glycero-3-phosphoethanolamine-N-[(polyethylene glycol)-2000] (DMPE-PEG);
1,2-Dimyristoyl-sn-glycero-3-phosphoethanolamine-N-[methoxy(polyethylene glycol)-2000] (DMPE-PEG-OCH₃);
1,2-Dimyristoyl-sn-glycero-3-phosphoethanolamine-N-[amino(polyethylene glycol)-2000] (DMPE-PEG-NH₂);
1,2-Dimyristoyl-sn-glycero-3-phosphoethanolamine-N-[carboxy(polyethylene glycol)-2000] (DMPE-PEG-COOH);
1,2-Dimyristoyl-sn-glycero-3-phosphoethanolamine-N-[maleimide(polyethylene glycol)-2000] (DMPE-PEG-maleimide);
1,2-Dimyristoyl-sn-glycero-3-phosphoethanolamine-N-[biotinyl(polyethylene glycol)-2000] (DMPE-PEG-Biotin);
1,2-Dilauroyl-sn-glycero-3-phosphoethanolamine-N-[(polyethylene glycol)-2000] (DLPE-PEG);
1,2-Dilauroyl-sn-glycero-3-phosphoethanolamine-N-[methoxy(polyethylene glycol)-2000] (DLPE-PEG-OCH₃);
1,2-Dilauroyl-sn-glycero-3-phosphoethanolamine-N-[amino(polyethylene glycol)-2000] (DLPE-PEG-NH₂);
1,2-Dilauroyl-sn-glycero-3-phosphoethanolamine-N-[carboxy(polyethylene glycol)-2000] (DLPE-PEG-COOH);
1,2-Dilauroyl-sn-glycero-3-phosphoethanolamine-N-[maleimide(polyethylene glycol)-2000] (DLPE-PEG-maleimide);
1,2-Dilauroyl-sn-glycero-3-phosphoethanolamine-N-[biotinyl(polyethylene glycol)-2000] (DLPE-PEG-Biotin);
1,2-dipalmitoyl-sn-glycero-3-phosphoethanolamine-N-[(polyethylene glycol)-2000] (DPPE-PEG);
1,2-dipalmitoyl-sn-glycero-3-phosphoethanolamine-N-[methoxy(polyethylene glycol)-2000] (DPPE-PEG-OCH₃);
1,2-dipalmitoyl-sn-glycero-3-phosphoethanolamine-N-[amino(polyethylene glycol)-2000] (DPPE-PEG-NH₂);
1,2-dipalmitoyl-sn-glycero-3-phosphoethanolamine-N-[carboxy(polyethylene glycol)-2000] (DPPE-PEG-COOH);
1,2-dipalmitoyl-sn-glycero-3-phosphoethanolamine-N-[maleimide(polyethylene glycol)-0.2000] (DPPE-PEG-maleimide); and
1,2-dipalmitoyl-sn-glycero-3-phosphoethanolamine-N-[biotinyl(polyethylene glycol)-2000] (DPPE-PEG-Biotin);
1,2-distearoyl-sn-glycero-3-phosphoethanolamine-N-[(polyacrylic acid)] (DSPE-PAA);
1,2-distearoyl-sn-glycero-3-phosphoethanolamine-N-[methoxy(polyacrylic acid)] (DSPE-PAA-OCH₃),
1,2-Distearoyl-sn-glycero-3-phosphoethanolamine-N-[amino(polyacrylic acid)] (DSPE-PAA-NH₂);
1,2-distearoyl-sn-glycero-3-phosphoethanolamine-N-[carboxy(polyacrylic acid)] (DSPE-PAA-COOH);
1,2-distearoyl-sn-glycero-3-phosphoethanolamine-N-[maleimide(polyacrylic acid)] (DSPE-PAA-maleimide);
1,2-distearoyl-sn-glycero-3-phosphoethanolamine-N-[biotinyl(polyacrylic acid)] (DSPE-PAA-biotin);
1,2-Dimyristoyl-sn-glycero-3-phosphoethanolamine-N-[(polyacrylic acid)] (DMPE-PAA);
1,2-Dimyristoyl-sn-glycero-3-phosphoethanolamine-N-[methoxy(polyacrylic acid)] (DMPE-PAA-OCH₃);
1,2-Dimyristoyl-sn-glycero-3-phosphoethanolamine-N-[amino(polyacrylic acid)] (DMPE-PAA-NH₂);
1,2-Dimyristoyl-sn-glycero-3-phosphoethanolamine-N-[carboxy(polyacrylic acid)] (DMPE-PAA-COOH);
1,2-Dimyristoyl-sn-glycero-3-phosphoethanolamine-N-[maleimide(polyacrylic acid)] (DMPE-PAA-maleimide);
1,2-Dimyristoyl-sn-glycero-3-phosphoethanolamine-N-[biotinyl(polyacrylic acid)] (DMPE-PAA-Biotin);
1,2-Dilauroyl-sn-glycero-3-phosphoethanolamine-N-[(polyacrylic acid)] (DLPE-PAA);
1,2-Dilauroyl-sn-glycero-3-phosphoethanolamine-N-[methoxy(polyacrylic acid)] (DLPE-PAA-OCH₃);
1,2-Dilauroyl-sn-glycero-3-phosphoethanolamine-N-[amino(polyacrylic acid)] (DLPE-PAA-NH₂);
1,2-Dilauroyl-sn-glycero-3-phosphoethanolamine-N-[carboxy(polyacrylic acid)] (DLPE-PAA-COOH);
1,2-Dilauroyl-sn-glycero-3-phosphoethanolamine-N-[maleimide(polyacrylic acid)] (DLPE-PAA-maleimide);
1,2-Dilauroyl-sn-glycero-3-phosphoethanolamine-N-[biotinyl(polyacrylic acid)] (DLPE-PAA-Biotin);
1,2-dipalmitoyl-sn-glycero-3-phosphoethanolamine-N-[(polyacrylic acid)] (DPPE-PAA).;
1,2-dipalmitoyl-sn-glycero-3-phosphoethanolamine-N-[methoxy(polyacrylic acid)] (DPPE-PAA-OCH₃);
1,2-dipalmitoyl-sn-glycero-3-phosphoethanolamine-N-[amino(polyacrylic acid)], (DPPE-PAA-NH₂);
1,2-dipalmitoyl-sn-glycero-3-phosphoethanolamine-N-[carboxy(polyacrylic acid)] (DPPE-PAA-COOH);
1,2-dipalmitoyl-sn-glycero-3-phosphoethanolamine-N-maleimide(polyacrylic acid)] (DPPE-PAA-maleimide); and
1,2-dipalmitoyl-sn-glycero-3-phosphoethanolamine-N-[biotinyl(polyacrylic acid)] (DPPE-PAA-Biotin).

More than one type of amphiphilic molecule may be in the disclosed FPdot, for example, the FPdot may comprise multiple amphiphilic molecules selected from the group consisting of 1,2-distearoyl-sn-glycero-3-phosphoethanolamino-N-[(polyethylene glycol)-2000]* (DSPE-PEG), 1,2-Dimyristoyl-sn-glycero-3-phosphoethanolamine N-[(polyethylene glycol)-2000]* (DMPE-PEG), 1,2-Dilauroyl-sn-glycero-3-phosphoethanolamine-N-[(polyethylene glycol)-2000]* (DLPE-PEG), 1,2-dipalmitoyl-sn-glycero-3-phosphoethanolamine-N-[(polyethylene glycol)-2000]* (DPPE-PEG), 1,2-distearoyl-sn-glycero-3-phosphoethanolamino-N-[amino(polyacrylic acid)]* (DSPE-PAA), 1,2-distearoyl-sn-glycero-3-phosphoethanolamino-N-[amino(polyacrylic acid)]* (DMPE-PAA), 1,2-distearoyl-sn-glycero-3-phosphoethanolamino-N-[amino(polyacrylic acid)]* (DLPE-PAA), 1,2-distearoyl-sn-glycero-3-phosphoethanolamino-N-[amino(polyacrylic acid)]* (DPPE-PAA) derivatives and combinations thereof, wherein * represents the point of attachment to the functional group.

The fluorescent conjugated polymer may be a polymer comprising a Π-electron delocalized backbone. The conjugated polymer may be partially or fully conjugated. The fluorescent conjugated polymer may be a chromophoric polymer which allows selective light absorption and results in characteristic colouration.

The fluorescent conjugated polymer may be a homopolymer, a blend of polymers or a copolymer. The fluorescent conjugated polymer may comprise repeating units of optionally substituted cyclic group linked to O-containing heteroaryl group.

The optionally substituted cyclic group may be monocylic or polycyclic. The optionally substituted cyclic group may be aromatic and may be selected from the group consisting of fluorene groups, phenylene groups, thiophene groups, carbazole groups, and boron-dipyrromethene groups.

The O-containing heteroaryl group may be monocylic or polycylic and may be selected from the group consisting of diazole, benzodiazole, oxazole, benzooxazole, pyran, furan, and benzofuran.

The fluorescent conjugated polymer may therefore be selected from the group consisting of fluorene polymers, phenylene vinylene polymers, phenylene polymers, phenylene ethynylene polymers, thiophen polymers, carbazole fluorene polymers, boron-dipyrromethene-based polymers, and polymer blends and copolymers thereof.

The fluorescent conjugated polymer may be poly(9,9-dihexylfluorene-alt-2,1,3-benzoxadiazole) (PFBD):

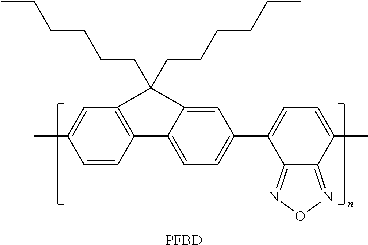

Advantageously, when PFBD is selected as the conjugated polymer, the absorption maximum of PFBD may advantageously perfectly match the 488 nm laser equipped on a confocal laser scanning microscope.

Advantageously, the extinction coefficient of the presently disclosed PFBD-containing FPdots at 488 nm may be 1.44×10⁸ M⁻¹ cm⁻¹, which may be superior to that of conventional conjugated polymer-based dots. For example, dots that contain poly(fluorene-alt-benzothiadiazole)(PFBT) may only display an extinction coefficient of 1×10⁷ M⁻¹ cm⁻.

Advantageously, the quantum yield of the presently disclosed PFBD-containing FPdots may be 32% while that of other PFBT-containing conjugated polymer dots is 30%. In one embodiment, the disclosed FPdots have a core-shell structure comprising a hydrophobic core and hydrophilic segments forming a protective shell layer.

In one embodiment, the disclosed FPdots may comprise a core and a shell, said core comprising the conjugated polymer, and said shell comprising the amphiphilic molecule.

The hydrophobic core of said FPdot may comprise the conjugated polymer and the hydrophilic shell may comprise the hydrophilic segment of the amphiphilic molecule. The hydrophobic segment of the amphiphilic molecule may be located within the hydrophobic core of the FPdot.

In one embodiment, the incorporation of DSPE-PEG to PFBD can form a core-shell structure where hydrophilic PEG segments provide the protective shell layer and hydrophobic PFBD and hydrophobic DSPE form the hydrophobic core.

In another embodiment, a biological molecule may be conjugated to the surface of the FPdot. The biological molecule may be selected from the group consisting of amino acids, peptides, polypeptides, nucleic acids, carbohydrates, lipids, fatty acids, antibodies, aptamers and proteins. The biological molecule may be conjugated to the surface of the FPdot via one or more abovementioned functional groups.

The biological molecule may be cell penetrating peptide which facilitates cellular uptake of various molecular cargo. The cell penetrating peptide may be derived from HIV-1 transactivator of transcription (Tat) protein. The cell penetrating peptide may be Tat (YRKKRRQRRRC) peptide.

In another embodiment, the biological molecule may be an antibody capable of targeting receptors over-expressed on a cell membrane. The targeted receptor may be human epidermal growth factor 2 (HER2). In this embodiment, human epidermal growth factor receptor 2 (HER2)-overexpressed breast cancer cells could be specifically targeted using anti-HER2 antibody-functionalized FPdots.

The way in which the biological molecule may be attached to the surface of the FPdots is dependent on the type of functional group located on said surface. For example, attachments of peptides to fluorescent polymer dot-NH₂ (FPdot-NH₂) or fluorescent polymer dot-COOH (FPdot-COOH) may be through a carboiimide-mediated coupling reaction. Attachments of peptides to fluorescent polymer dot-maleimide (FPdot-maleimide) may be through thiol-maleimide click reaction. Advantageously, the properties of biomolecule-conjugated functionalized-FPdots may not change upon bioconjugation.

The weight ratio of conjugated polymer to amphiphilic molecule may be in the range of about 25% to about 75%, about 25% to about 70%, about 25% to about 65%, about 25% to about 60%, about 25% to about 55%, about 25% to about 50%, about 25% to about 45%, about 25% to about 40%, about 25% to about 65%, about 25% to about 30%, about 30% to about 75%, about 35% to about 75%, about 40% to about 75%, about 45% to about 75%, about 50% to about 75%, about 55% to about 75%, about 60% to about 75%, about 65% to about 75%, or about 70% to about 75%. The weight ratio of conjugated polymer to amphiphilic polymer may be about 25%, about 30%, about 35%, about 40%, about 45%, about 50%, about 55%, about 60%, about 65%, about 70%, about 75%. The weight ratio of conjugated polymer to amphiphilic molecule may be about 50%.

The size of the FPdot may be in the range of about 30 nm to about 50 nm, about 30 nm to about 45 nm, 30 nm to about 40 nm, 30 nm to about 35 nm, 35 nm to about 50 nm, nm to about 50 nm, or 45 nm to about 50 nm. The size of the FPdot may be about 30 nm, about 35 nm, about 40 nm, about 45 nm, or about 50 nm.

### Methods of Synthesizing Fluorescent Polymer Dots

In another aspect, there is provided a method for preparing a fluorescent polymer dot, the method comprising the steps of:

a) preparing a mixture of fluorescent conjugated polymer and amphiphilic molecule in an aprotic solvent; and

b) adding said mixture to a protic solvent, to form the polymer dot comprising one or more fluorescent conjugated polymers forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties.

The aprotic solvent may be selected from the group consisting of tetrahydrofuran, ether, dichloromethane, acetone, acetonitrile, DMF, and the mixtures thereof. The aprotic solvent may be tetrahydrofuran, acetone, or mixtures thereof.

The protic solvent may be selected from lower alcohols, water, and mixtures thereof. The protic solvent may be selected from the group consisting of methanol, ethanol, propanol, butanol and MilliQ water.

The FPdots may have a spherical shape which may be formed when a mixture of conjugated polymer and amphiphilic molecule in aprotic solvent is added to a protic solvent. The sudden decrease of solvent hydrophobicity may lead to the collapse of hydrophobic segments of the amphiphilic molecule and conjugated polymer. The FPdots may therefore form a spherical shape with hydrophobic segments at the core and hydrophilic segments of the amphiphilic molecule as the shell.

Advantageously, the size of the FPdots may be controlled by the disclosed methods.

In one embodiment, the method further comprises the step of controlling the size of the FPdots produced. The step may comprise modifying the initial concentration of conjugated polymer in aprotic solvent.

The initial concentration of conjugated polymer in aprotic solvent may be in the range of about 0.05 mg/mL to about 1.50 mg/mL, about 0.10 mg/mL to about 1.50 mg/mL, about 0.20 mg/mL to about 1.50 mg/mL, about 0.30 mg/mL to about 1.50 mg/mL, about 0.40 mg/mL to about 1.50 mg/mL, about 0.50 mg/mL to about 1.50 mg/mL, about 0.60 mg/mL to about 1.50 mg/mL, about 0.70 mg/mL to about 1.50 mg/mL, about 0.80 mg/mL to about 1.50 mg/mL, about 0.90 mg/mL to about 1.50 mg/mL, about 1.00 mg/mL to about 1.50 mg/mL, about 1.10 mg/mL to about 1.50 mg/mL, about 1.20 mg/mL to about 1.50 mg/mL, about 1.30 mg/mL to about 1.50 mg/mL, about 1.40 mg/mL to about 1.50 mg/mL, about 0.05 mg/mL to about 1.40 mg/mL, about 0.05 mg/mL to about 1.30 mg/mL, about 0.05 mg/mL to about 1.20 mg/mL, about 0.05 mg/mL to about 1.10 mg/mL, about 0.05 mg/mL to about 1.00 mg/mL, about 0.05 mg/mL to about 0.90 mg/mL, about 0.05 mg/mL to about 0.80 mg/mL, about 0.05 mg/mL to about 0.70 mg/mL, about 0.05 mg/mL to about 0.60 mg/mL, about 0.05 mg/mL to about 0.50 mg/mL, about 0.05 mg/mL to about 0.40 mg/mL, about 0.05 mg/mL to about 0.30 mg/mL, about 0.05 mg/mL to about 0.20 mg/mL, or about 0.05 mg/mL to about 0.10 mg/mL. The initial concentration may be about 0.05 mg/mL, about 0.10 mg/mL, about 0.20 mg/mL, about 0.30 mg/mL, about 0.40 mg/mL, about 0.50 mg/mL, about 0.60 mg/mL, about 0.70 mg/mL, about 0.80 mg/mL, about 0.90 mg/mL, about 1.00 mg/mL, about 1.10 mg/mL, about 1.20 mg/mL, about 1.30 mg/mL, about 1.40 mg/mL, or about 1.50 mg/mL.

In one embodiment, the weight ratio of conjugated polymer to amphiphilic molecule is maintained although the initial concentration of conjugated polymer is varied.

By being able to control the size of the disclosed FPdots, the fluorescence quantum yield of the FPdots may be controlled by tuning the initial conjugated polymer concentration during synthesis. This is highly advantageous as the fluorescence quenching of fluorescent polymer dots is a well-known obstacle to obtain fluorescent polymer dots with high quantum yield.

### Bioconjugation of Fluorescent Polymer Dots

The fluorescent polymer dots may be conjugated with one or more biomolecules. As mentioned above, the disclosed fluorescent polymer dots may be customizable which allows a wide variety of biomolecules to be conjugated to said fluorescent polymer dot allowing a for a variety of bioimaging applications.

In another aspect, a method of detecting a target molecule in a biological sample, the method comprising contacting a biological sample with a disclosed fluorescent polymer dot.

Advantageously, the disclosed fluorescent polymer dots may be useful in multimodal imaging applications, such as fluorescence and magnetic resonance imaging.

Advantageously, the disclosed fluorescent polymer dots may display superior living cell tracing ability up 10, to about 12 generations.

### BRIEF DESCRIPTION OF DRAWINGS

The accompanying drawings illustrate disclosed embodiments and serves to explain the principles of the disclosed embodiments. It is to be understood, however, that the drawings are designed for purposes of illustration only, and not as a definition of the limits of the invention.

FIG. 1 depicts the chemical structures of PFBD, DSPE-PEG-OCH₃, DSPE-PEG-NH₂, DSPE-PEG-COOH and DSPE-PEG-Maleimide.

FIG. 2a is a graph showing the size distribution of FPdots prepared from PFBD in THF solution at an initial concentration of 1.00 mg/mL.

FIG. 2b is a graph showing the UV-vis absorption and PL emission spectra of FPdots prepared from PFBD in THF solution at an initial concentration of 1.00 mg/mL.

FIG. 3a is a graph showing the size distribution results of PFBD FPdots prepared with different initial PFBD concentrations (1.00 mg/mL, 0.50 mg/mL, 0.20 mg/mL and 0.10 mg/mL) in THF solution.

FIG. 3b is a graph showing the quantum yields of PFBD FPdots prepared from different initial PFBD concentrations (1.00 mg/mL, 0.50 mg/mL, 0.20 mg/mL and 0.10 mg/mL) in THF solution.

FIG. 4 is a series of transmission electron microscope (TEM) images of FPdots prepared from initial PFBD concentrations in THF solution: (a) 1.00 mg/mL, (b) 0.50 mg/mL and (c) 0.25 mg/mL.

FIG. 5 shows a series of size distribution histograms and HR-TEM images of PFBD FPdots with surface (a) amine (FPdot-NH2); (b) carboxyl (FPdot-COOH); and (c) maleimide (FPdot-Maleimide) functional groups.

FIG. 6 shows a graph depicting the time course of fluorescence intensity change of 2 nM FPdots when compared with Qtracker® 585 in cell culture medium at 37° C.

FIG. 7 is a series of flow cytometry histograms of MCF-7 breast cancer cells after incubation with 2 nM Tat-PFBD-containing FPdots obtained from (a) FPdot-Maleimide; (b) FPdot-NH₂; and (c) FPdot-COOH; and (d) 2 nM Qtracker® 585.

FIG. 8 shows a series of confocal images of MCF-7 breast cancer cells after incubation with 2 nM Tat-PFBD-containing FPdots obtained from (a) FPdot-Maleimide; (b) FPdot-NH₂; (c) FPdot-COOH; and (d) 2 nM Qtracker® 585.

FIG. 9 shows a graph depicting the photobleaching resistance of Tat-FPdot and Qtracker® 585 to the continuous irradiation by a laser beam (1.25 mW) at 488 nm.

FIG. 10 is a graph showing cell viability data of MCF-7 cells after 48 h incubation with FPdots and Tat-FPdot obtained from FPdot-Maleimide, FPdot-NH₂ and FPdot-COOH, respectively.

FIG. 11 is a schematic diagram showing the route from FPdot synthesis to its use in cell tracing.

### EXAMPLES

Non-limiting examples of the invention and comparative examples will be further described in greater detail by reference to specific Examples, which should not be construed as in any way limiting the scope of the invention.

### Experimental

### Materials

Tat (YRKKRRQRRRC), 1,2-distearoyl-sn-glycero-3-phosphoethanolamine-N-[methoxy(polyethylene glycol)-2000] (DSPE-PEG-OCH₃), 1,2-distearoyl-sn-glycero-3-phosphoethanolamine-N-[carboxy(polyethylene glycol)-2000] (DSPE-PEG-COOH), 1,2-Distearoyl-sn-glycero-3-phosphoethanolamine-N-[amino(polyethylene glycol)-2000] (DSPE-PEG-NH₂), 1,2-distearoyl-sn-glycero-3-phosphoethanolamine-N-[maleimide(polyethylene glycol)-2000] (DSPE-PEG-Maleimide) were purchased from Avanti Polar Lipids, Inc., Qtracker™ 585 was purchased from Life Technologies, Invitrogen, Singapore. Fetal bovine serum (FBS) was purchased from Gibco (Lige Technologies, Ag, Switzerland). 3-(4,5-Dimethylthiazol-2-yl)-2,5-diphenyl tetrazolium bromide (MTT) and penicillin-streptomycin solution were purchased from Sigma-Aldrich. Dulbecco's modified essential medium (DMEM) was a commercial product of National University Medical Institutes (Singapore). Phosphate-buffer saline (PBS; 10×) buffer with pH 7.4 (ultrapure grade) is a commercial product of first BASE Singapore. Alexa Fluor 488 was purchased from Life Technologies (Singapore). Fluorescein isothiocyanate (FITC) was purchased from Sigma-Aldrich (Singapore). Milli-Q Water (18.2 MΩ) was used to prepare the buffer solutions from the 10×PBS stock buffer. PBS (1×) contains NaCl (137 mM), KCl (2.7 mM), Na₂HPO₄ (10 mM) and KH₂PO₄ (1.8 mM).

### Characterization

The fluorescence spectra of the FPdots in water suspensions were measured using a fluorometer (LS-55, Perkin Elmer, USA) with an excitation wavelength of 471 nm for PFBD FPdots. Average particle size and size distribution of the FPdots were determined by LLS with a particle size analyzer (90 Plus, Brookhaven Instruments Co. USA) at a fixed angle of 900 at room temperature. The morphology of PFBD-containing FPdots was also studied by TEM (JEM-2010F, JEOL, Japan).

### Cell Culture

MCF-7 breast cancer cells were cultured in Dulbecco's Modified Eagle Medium (DMEM) containing 10% fetal bovine serum and 0.1% penicillin streptomycin at 37° C. in a humidified environment containing 5% CO₂. Before experiments, the cells were pre-cultured well until confluence was reached.

### Confocal Imaging and Cell Tracing

MCF-7 breast cancer cells were cultured in a six-well plate (Costar, IL, USA) at 37° C. After 80% confluence, the medium was removed, and the adherent cells were washed twice with 1×PBS buffer. Tat-FPdots obtained from FPdot-COOH in DMEM medium containing 10% fetal bovine serum and 1% penicillin streptomycin were then added to the wells. After overnight incubation at 37° C., the cells were washed twice with 1×PBS buffer and detached by 1× trypsin. DMEM culture medium was then added into the cells-contained trypsin solutions. After centrifugation at 1500 rpm for 5 min, the precipitated cells were resuspended in new culture medium. Upon dilution, the cells were subcultured in six-well plates containing cell culture coverslips for day 1, 3, 5, and 7 tracing and imaging. After designated time intervals, the cells were washed twice with 1×PBS buffer, and the cell culture coverslip was picked out and fixed by 75% filtered ethanol for 20 min, which was further washed twice with 1×PBS buffer, and sealed with mounting medium and imaged by CLSM (Zeiss LSM 410, Jena, Germany) with imaging software (Fluoview FV1000). The rest cells in the same well were trypsinalized to suspend in 1× PBS buffer. The corresponding fluorescence intensities of cells were then analyzed by flow cytometry measurements using Cyan-LX (DakoCytomation) and the histogram of each sample was obtained by counting 10,000 events (hex=488 nm, 580/20 nm bandpass filter). The cell tracing studies using Qtracker™ 585 and Tat-bioconjugated FPdot from FPdot-NH₂ and FPdot-Maleimide followed the same procedures.

### Cytotoxicity Study

MTT assays were performed to access the metabolic activity of MCF-7 breast cancer cells. MCF-7 cells were seeded in 96-well plates (Costar, Chicago, Ill.) at an intensity of 4×104 cells/mL. After 24 h of incubation, the medium was replaced by FPdot suspensions at a concentration of 2, 4 and 10 nM for further 48 h incubation, respectively. The wells were then washed twice with 1×PBS buffer, and freshly prepared MTT (100 μL, 0.5 mg/mL) solution in culture medium was added to each well. The MTT medium solution was carefully removed after 3 h of incubation in the incubator. Filtered DMSO (100 μL) was then added into each wells, and the plate was gently shaken for 10 min at room temperature to dissolve all precipitation formed. The absorbance of MTT at 570 nm was monitored by the microplate reader (Genios Tecan). Cells viability was expressed by the ratio of absorbance of the cells incubated with dot suspension to that of cells incubated with culture medium only. The cell viability of Tat-bioconjugated FPdot obtained from FPdot-COOH, FPdot-NH₂ and FPdot-Maleimide was also investigated following the same procedures as described.

### Photostability Study

To study the photostability of FPdots, confocal images of MCF-7 cancer cells incubated with Tat-bioconjugated FPdot were taken at 2 min time interval under continuous laser excitation at 488 nm (1.25 mW). The fluorescence intensity of each image was assessed by ImageJ software. Moreover, the photostability of Qtracker™ 585, Alexa Fluor 488 and fluorescein isothiocyanate in MCF-7 cancer cells were also studied at the same conditions. The photostability of each probe was expressed by the ratio of fluorescence intensity upon continuous laser scanning for different time intervals to the initial value as a function of exposure time.

### Example 1

### Synthesis of Poly(9,9-dihexylfluorene-alt-2,1,3-benzoxadiazole) (PFBD)

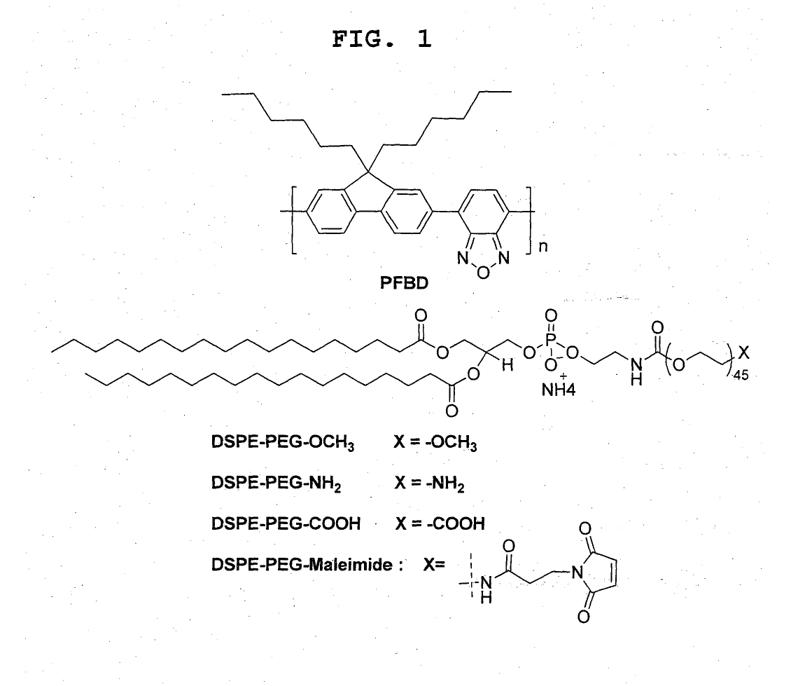

Poly(9,9-dihexylfluorene-alt-2,1,3-benzoxadiazole) (PFBD) was synthesized by palladium-catalyzed Suzuki polycondensation reaction between 4,7-dibromo-2,1,3-benzoxadiazole and 2,7-bis(1,3,2-dioxaborinane)-9,9-dihexylfluorene (FIG. 1).

A Schlenk tube fitted with a condenser was charged with 2,1,3-benzoxadiazole (4.0 g, 33.4 mmol) and iron (0.4 g, 7.1 mmol). The mixture was heated to 90° C. and bromine (5 mL, 98.2 mmol) was added drop-wise. After continuous stirring at 90° C. for another 2 h, the mixture was cooled to room temperature and poured into 500 mL of water. The obtained solid was filtered, dropped into 300 mL of saturated NaHCO₃, and stirred for 1 h to ensure complete neutralization. The crude product was further purified by silica gel column chromatography (hexane/dichloromethane=9/1) to give 4,7-dibromo-2,1,3-benzoxadiazole (6.5 g, yield: 70.9%) as a white solid. 1HNMR (500 MHz, CDCl₃, ppm) δ: 7.51 (s, 2H). 13CNMR (125 MHz, CDCl₃, ppm) δ: 149.37, 134.22, 108.69.

A Schlenk tube was charged with 4,7-dibromo-2,1,3-benzoxadiazole (139.0 mg, 0.5 mmol), 2,7-bis(1,3,2-dioxaborinane)-9,9-dihexylfluorene (251.0 mg, 0.5 mmol) and Pd(PPh₃)₄ (12.5 mg, 0.01 mmol) in toluene (20 mL). The tube was then sealed with a rubber septum and degassed with three freeze-pump-thaw cycles to remove air. Upon heating to 80° C., an aqueous Et₄NOH solution (20 wt %, 2 mL) was added to initiate the reaction. After 20 h, the reaction was stopped and cooled down to room temperature. The mixture was then dropped slowly into methanol (100 mL) to precipitate the crude product, followed by centrifugation. The crude polymer was subsequently dissolved in dichloromethane (200 mL), washed with water 3 times and dried by MgSO₄. After solvent removal, PFBD was obtained as an orange solid (163.6 mg, yield: 72%). 1H NMR (500 MHz, CDCl₃, ppm) δ: 8.14 (br, 4H), 7.96 (br, 2H), 7.84 (br, 2H), 2.19 (br, 4H), 1.14 (br, 12H), 0.87 (br, 4H), 0.78 (br, 6H). Gel permeation chromatography (GPC) result suggests that the obtained PFBD has a weight-average molecular weight (Mw) of 12,000 g mol⁻¹ with a polydispersity index (PDI) of 1.8.

### Example 2a

### General Preparation of Fluorescent Polymer Dots

To obtain a well-dispersed FPdot suspension in water, a modified nano-precipitation was used involving a rapid addition of a tetrahydrofuran (THF) solution of hydrophobic conjugated polymer and amphiphilic matrix to an excess volume of MilliQ water. The sudden decrease of solvent hydrophobicity led to the collapse of hydrophobic segments of matrix and conjugated polymer. Therefore, the FPdots formed in a spherical shape with a core comprising conjugated polymer and hydrophobic segments of the matrix and a shell comprising hydrophilic segments of the matrix.

To demonstrate the FPdots formation, DSPE-PEG was used as the matrix ° and PFBD as the conjugated polymer. PFBD was synthesized as in Example 1. DSPE-PEG contains two hydrophobic alkyl chains and one hydrophilic polyethylene glycol PEG chain (MW=2000). As a result, the alkyl chains interacted with PFBD to form a hydrophobic core, while the PEG chain provided hydrophilic functional groups extending towards the water phase.

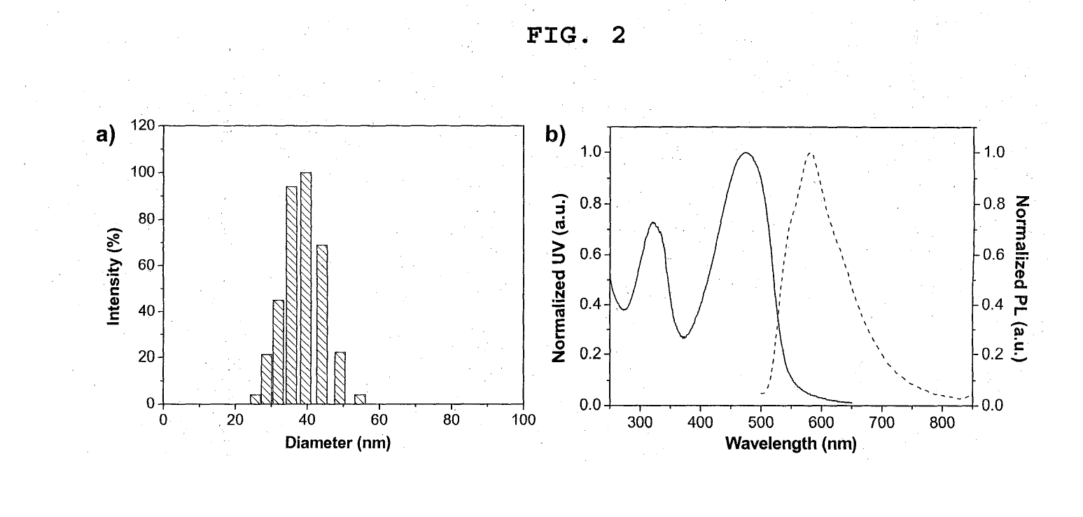

The average effective diameter of FPdots was measured by dynamic light scattering (DLS) at a PFBD concentration of 4 μg/mL in aqueous solution. The FPdots had an effective diameter of 40 nm with narrow distribution (FIG. 2). In addition, the DLS results suggested that the size of the FPdots did not change after storage for over three months, elucidating the good stability of FPdots in aqueous solutions. The unchanged particle sizes are believed to be contributed by the presence of hydrophilic PEG chains, which formed the protective shell layer outside the PFBD core and thus prevented the FPdots from aggregation.

### Example 2b

### Detailed Preparation of Fluorescent Polymer Dots

The FPdots were prepared via a modified nanoprecipitation method. A THF solution (1 mL) containing 1 mg of PFBD and 2 mg of DSPE-PEG was injected into 10 mL MilliQ water, followed by sonication of mixture for 90 s at 18 W output using a microtip probe sonicator (XL2000, Misonix Incorporated, NY). The mixture was then stirred at room temperature in the dark and overnight to evaporate 0.5 the THF. The formed FPdot suspension was then filtered through a 200 nm membrane filter.

Different concentration PFBD-containing FPdots were also prepared following the same procedure with corresponding different initial concentration of PFBD in THF solutions (0.50, 0.25, 0.20, and 0.10 mg/mL). FPdots with different surface functional groups were prepared following the same procedure but with a PFBD, DSPE-PEG and DSPE-PEG-X (X represents various functional groups, such as methoxy, amino, carboxylic, maleimide, or biotin groups) at weight ratio 1:1:1.

### Example 3

### Characterization of Fluorescent Polymer Dots

The absorption and emission maxima of the PFBD-containing FPdots were located at 471 and 583 nm, respectively. The fluorescence quantum yield was measured to be 17.1%. On the contrary, precipitation of PFBD itself from THF to water only gave a fluorescence quantum yield of 2%. The low quantum yield was attributed to polar media induced fluorescence quenching as PFBD possesses intramolecular charge transfer (ICT) characteristics due to a strong electron-deficient benzoxadiazole unit. The incorporation of DSPE-PEG to PFBD formed a core-shell structure where hydrophilic PEG segments provided the protective shell layer, preventing the PFBD core from the surrounding aqueous solution.

### Example 4

### Optimization and Characterization of Resulting Fluorescent Polymer Dots

The size, morphology and properties of FPdots were manipulated by adjusting the FPdot synthetic process.

Different initial concentrations of PFBD in THF solution from 1.00 to 0.10 mg/mL (1.00 0.50, 0.25 0.20 and 0.10 mg/mL) via a ten-fold dilution into MilliQ water was used while retaining the DSPE-PEG to PFBD weight ratio.

The effects of initial PFBD concentration changes to the optical properties of final FPdots were also evaluated.

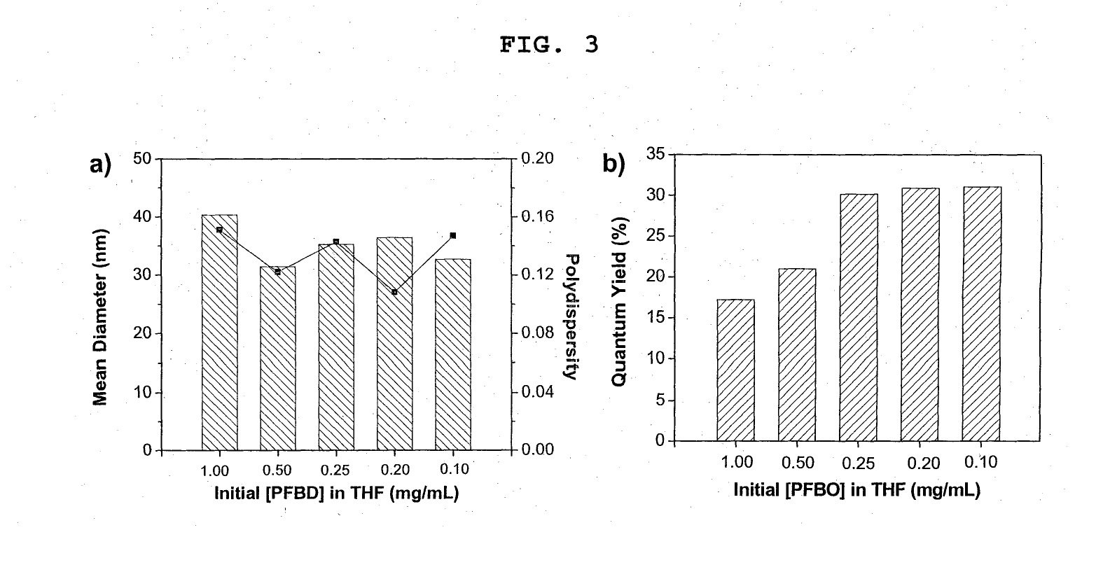

It was observed that upon decrease of the initial concentration of PFBD from 1.00 to 0.25 mg/mL, the fluorescence quantum yield showed more than an 80% increase from 17% to 31.7% (FIG. 3b). Further decrease of the initial concentration of PFBD did not change the quantum yield of the FPdots.

To further investigate the relationship between optical properties and initial concentration of the precursors in FPdots synthesis, the morphologies of different batches of FPdots were further investigated.

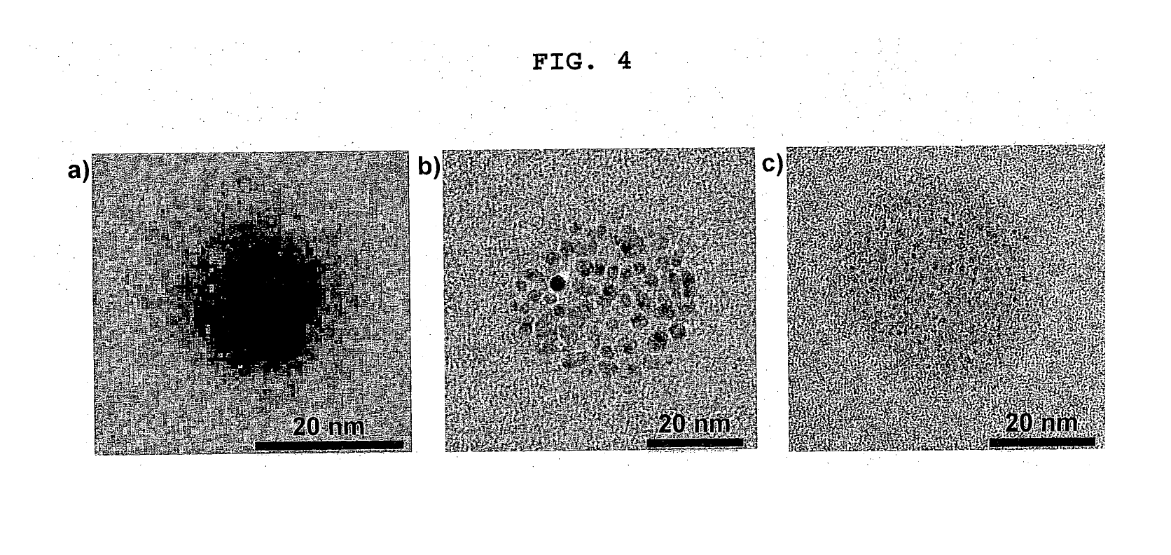

The sizes of FPdots prepared from different initial PFBD concentrations were firstly measured by DLS. As shown in FIG. 3, the DLS measurement revealed that the mean diameter of FPdots decreased from 40 to 30 nm with polydispersities around 0.15 when the initial PFBD concentration was decreased from 1.00 to 0.10 mg/mL. The morphology studies using TEM revealed almost unchanged FPdots sizes with diameters around 32±7 nm for all the samples. Although the size increased with increasing PFDB initial concentration, the morphologies of these FPdots were quite different from each other. For instance, the FPdots obtained from 1.0 mg/mL of PFBD in THF mixture exhibited a typical core-shell architecture with compact dark core around ˜20 nm surrounded by ˜7 nm gray shell (FIG. 4a). As for FPdots prepared from 0.50 mg/mL of PFBD in THF initial concentration, the core-shell structure no longer existed, instead of which, dark spots with ˜6 nm doped micelle dots uniformly distributed in the most interior area of the sphere were observed (FIG. 4b). When the initial PFBD concentration in THF dropped to 0.25 mg/mL or lower, similar punctate pattern of dark dots were observed in the FPdot interior, while the PFBD dark dots were much smaller with sizes around 2-3 nm (FIG. 4C). The molecular weight of PFBD used was around ˜25000 g/mole and assuming that the single PFBD chain would collapse into a compact spherical dots with the density of ˜1.0 g/mL, the number of chains within each dark dot could be calculated. The calculated numbers of chains in the dark core of dogs were ˜100 (20 nm PFBD core prepared from initial PFBD concentration of 1.00 mg/mL), ˜4 (6 nm PFBD spots prepared from initial PFBD concentration of 0.50 mg/mL) and ˜1 (2.3 nm PFBD dots prepared from initial PFBD concentration of 0.25 mg/mL or lower), respectively.

The formation of FPdots was driven by the hydrophobicity change of solvents, where the hydrophobic segment collapsed with, each other to form the compact core. Meanwhile, the hydrophilic segments formed the protective shell outside of the hydrophobic core, preventing them from further aggregation or collapse. At a high initial concentration of PFBD and DSPE-PEG in THF via ten-fold dilution in water, the chance of PFBD chains collapsing with themselves was much higher than collapsing with DSPE, due to the much higher hydrophobicity of PFBD over DSPE. Thus, the PFBD chains were less likely to be separated by DSPE and tended to form a single dark core surrounded by a medium DSPE layer. At a low initial PFBD concentration condition, PFBD and DSPE-PEG were more dispersed in THF and water during the dilution and the possibility of PFBD chains to collapse with themselves decreased, which relatively increased the chance of DSPE meeting and encapsulating PFBD, thereby blocking further aggregation of PFBD. Therefore, PFBD presented as small dots patterned in the interior of the dots. When initial PFBD concentration in THF was decreased, the self-aggregation of PFBD chains was suppressed, resulting in less inter- and intra-molecule chain charge transfer that leads to fluorescence quenching. As a result and advantageously, the increased quantum yield is ascribed to the suppressed ICT effects.

### Example 5

### Surface Functionalization of PFBD-Containing FPdots

FPdots with different surface functional groups were synthesized through the same modification method as disclosed above. To demonstrate the generality of this strategy in preparation of stable and bright FPdots with different surface functional groups for long-term cell tracing application, DSPE-PEG-NH₂, DSPE-PEG-COOH and DSPE-PEG-Maleimide were mixed with DSPE-PEG at weight ratio of 1:1 as the matrix, respectively. An initial concentration of PFBD in THF at 0.25 mg/mL was used to prepare the FPdots.

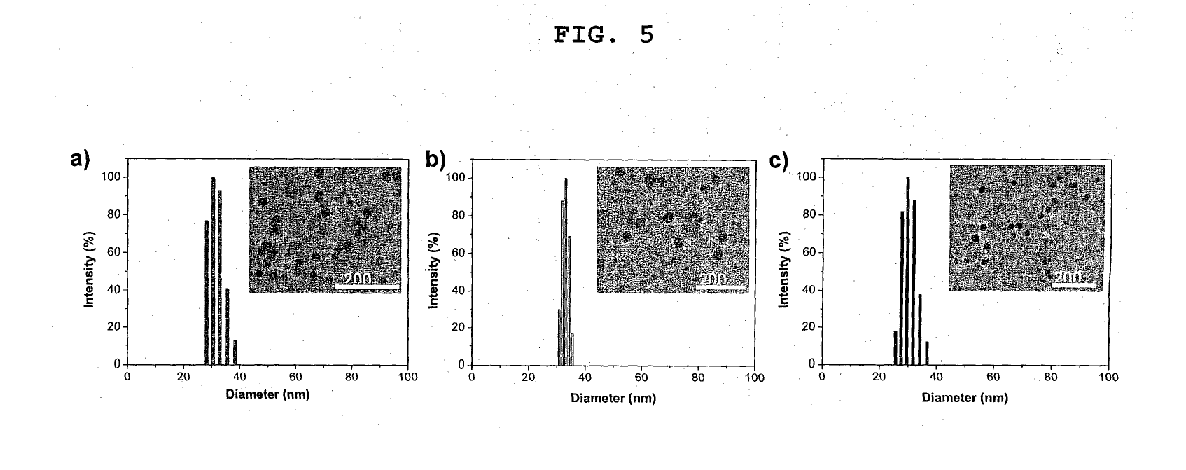

It was shown that the size and morphologies of these FPdots were unaffected upon the alteration of the DSPE-PEG derivatives (FIG. 5). A cell penetrating peptide derived from HIV-1 transactivator of transcription (Tat) protein, Tat (YRKKRRQRRRC) peptide was subsequently conjugated to the FPdots surface. The attachment of Tat to the surface of FPdot-NH₂ or FPdot-COOH were carried out through a carbodiimide-mediated coupling reaction, while the conjugation of Tat to FPdot-Maleimide was carried out via a thiol-maleimide click reaction. The properties of Tat-FPdots did not change upon bioconjugation.

FPdot-COOH (4 mL, 0.025 mg/mL) was reacted with 1.5 times of Tat (3.30 μL, 0.02M) with 10 times of 1-ethyl-3-(3-dimethylaminopropyl)carbodiimide (EDAC) as the sensitizer (84.2 μL, 1 mg/mL) overnight under stirring at room temperature. The unreacted Tat and EDAC were then removed through dialysis by 6000-8000 kDa membrane for three days. The sample was then freeze dried to 1 mL solution (0.1 mg/mL). The same procedure was followed for the Tat functionalization of FPdot-NH₂. As for Tat functionalization of FPdot-Maleimide, the FPdots (4 mL, 0.025 mg/mL) were simply mixed with Tat (3.20 μL, 0.02M) suspension for stirring overnight at room temperature. The excess Tat peptides were removed through dialysis.

### Comparative Example 1

### Characterization of Tat-Conjugated FPdots Versus Commercial Qtracker™

The molar extinction coefficient of the Tat-conjugated FPdots measured at absorption maximum at 471 nm is 1.44×10⁸ M⁻¹ cm⁻¹, which is ˜140 folds more than that of commercially available cell tracing probe, Qtracker™ 585 (1×10⁶ M⁻¹ cm⁻¹). The fluorescence quantum yield of Qtracker™ 585 upon excitation at 471 nm was measured to be 25.6%, which was also less superior to the presently disclosed Tat-conjugated FPdots (31.7%).

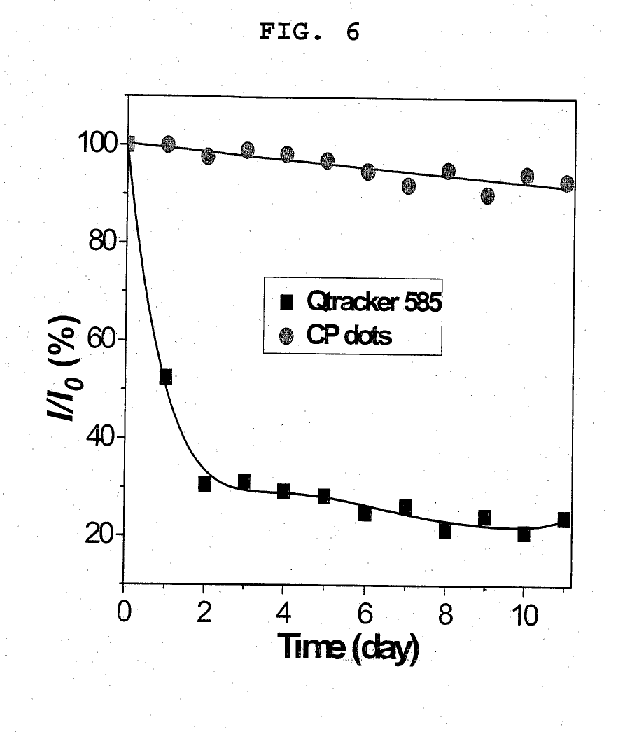

For long-term cell tracing applications, cell tracing probes should possess excellent fluorescence stability in it's biological environment to ensure accurate deciphering of the tracing results. The fluorescence stability of the Tat-conjugated FPdots in the cell culture medium (Dulbecco's Modified Eagle Medium, DMEM, supplemented with 10% fetal bovine serum, FBS) was evaluated at 37° C. As shown in FIG. 6, the Tat-conjugated FPdots remained at 92% of its fluorescence after 11-days of incubation in the medium. On the contrary, Qtracker™ 585 lost 48% of its fluorescence after 24 h of incubation, and was at 25% of its initial fluorescence after 11 days. The Tat-conjugated FPdots exhibited superior performance in molar extinction coefficient, fluorescence quantum yield, and long-term stability over Qtracker™ 585.

### Comparative Example 2

### In Vitro Cell Tracing and Imaging of Tat-Conjugated FPdots Versus Commercial Qtracker™

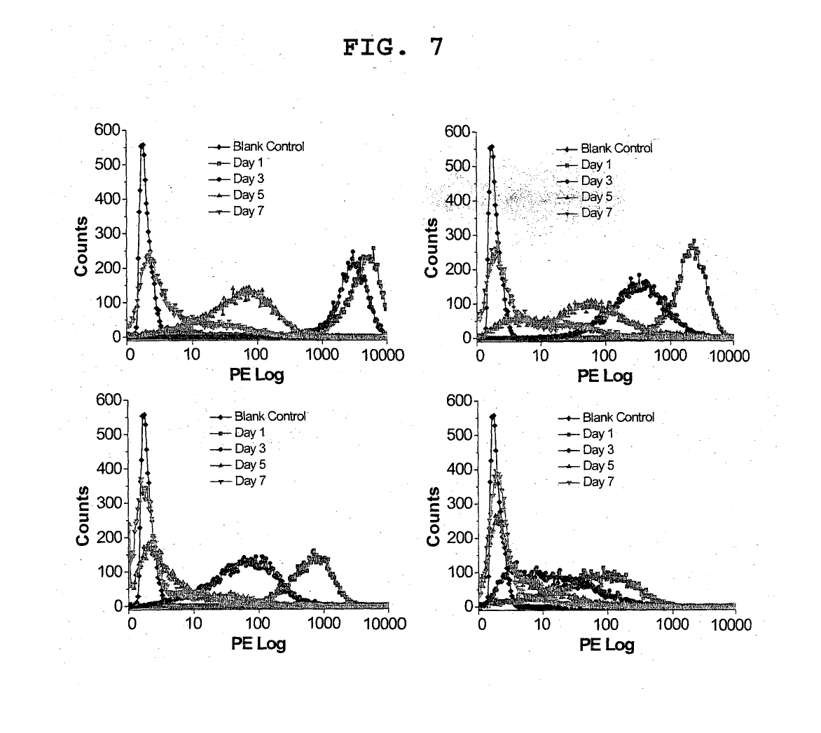

MCF-7 breast cancer cells were chosen as the cell lines, to demonstrate the cell tracing ability of Tat-CP dots, whose performance was compared with Qtracker™ 58.5 under the same experiment conditions. The MCF-7 cancer cells were first incubated with Tat-conjugated FPdots or Qtracker™ 585 at FPdot concentration of 2 nM overnight at 37° C. The labeled cells were then subcultured for cell reproduction and generation for designated time intervals (day 1, 3, 5 and 7). The fluorescence profiles of labelled cells were recorded using flow cytometry by counting 10,000 events (λex=488 nm, 580/20 nm bandpass filter), as shown in FIG. 7. Table 1 shows the labeling rate of MCF-7 cells. Tat-conjugated FPdots obtained from FPdot-Maleimide displayed the highest labeling efficiency for all the designated times while that obtained from FPdot-NH₂ gave comparable results, suggesting that labeling efficiencies were still above 80% after 5 days of incubation.

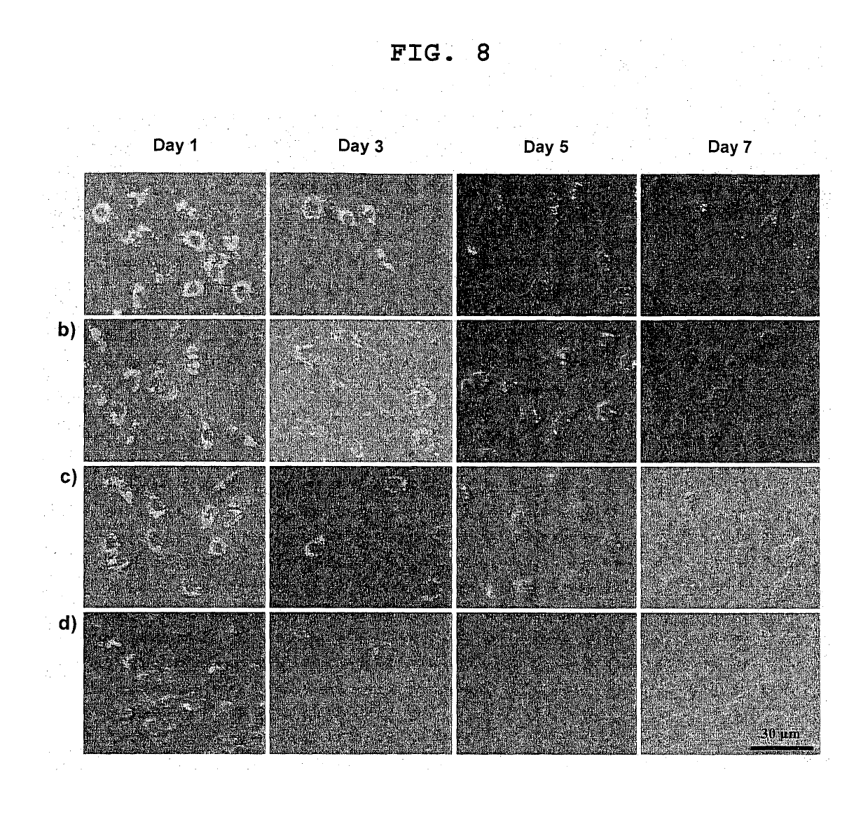

On the contrary, the labeling efficiency of MCF-7 cells after incubation with Qtracker™ 585 decreased to 26% after 5 days of incubation, which was less superior to Tat-conjugated FPdots obtained from FPdot-COOH which had the poorest performance amongst all the obtained Tat-conjugated FPdots. The flow cytometry results clearly revealed the excellent long-term in vitro cell tracing ability of Tat-conjugated FPdots over Qtracker™ 585. Confocal microscopy imaging was also performed to further confirm the cell tracing capability of Tat-conjugated FPdots. As shown in the FIG. 8, the fluorescence difference between MCF-7 cells labeled by Qtracker™ 585 and Tat-conjugated FPdots was observed after just one day of labeling, where Tat-conjugated FPdots treated MCF-7 cells all displayed much brighter green fluorescence than Qtracker™ 585 treated cells did. It should be noted that the Tat-conjugated FPdots were internalized into cell cytoplasm and uniformly distributed outside nuclei due to their larger sizes when compared to nuclear pore (˜9 nm). After 3 days of incubation, the morphology of cells treated by Tat-conjugated FPdots was still distinguishable from the bright fluorescence, while only punctate fluorescence was observed for Qtracker™ 585 stained cells. The fluorescence of Qtracker™ 585 in MCF-7 cells faded out at day 5 after incubation, while the signal of cells labeled by Tat-conjugated FPdots from FPdot-Maleimide is still distinguishable at day 7 after incubation. Therefore, Tat-conjugated FPdots can serve as an ultrabright and ultrastable fluorescent probes in long-term cell tracing and imaging applications.

TABLE 1

| | Labeling Efficiency (%) | | | |
|---|---|---|---|---|
| | FN-Maleimide- | FN—NH2- | FN—COOH- | Qtracker |
| Day | TAT | TAT | TAT | 585 |
| 1 | 99.38 | 99.21 | 98.83 | 84.08 |
| 3 | 98.21 | 98.26 | 96.62 | 43.97 |
| 5 | 82.19 | 80.83 | 50.77 | 26.89 |
| 7 | 31.12 | 29.50 | 13.14 | 4.31 |

### Comparative Example 3

### Cytotoxity and Photostability of Tat-Conjugated FPdots Versus Commercial Qtracker™

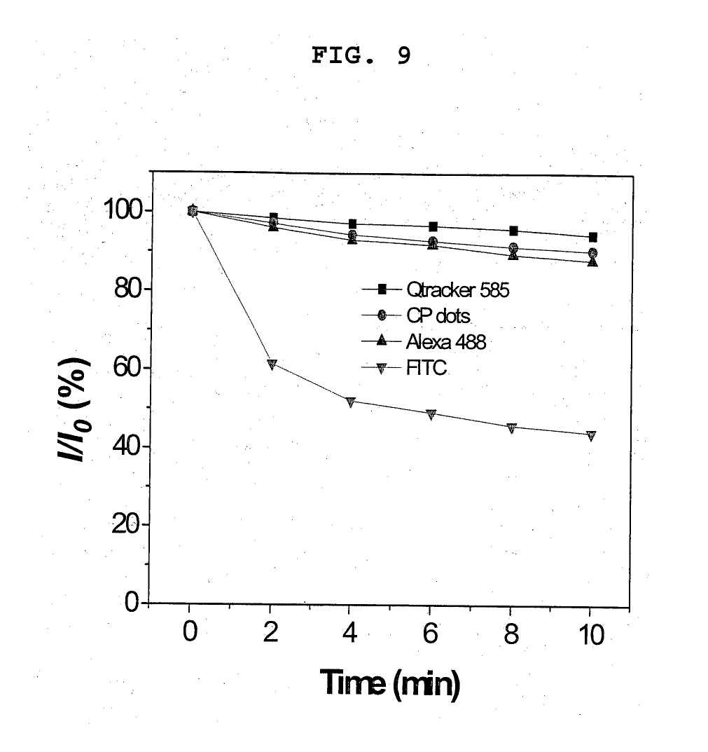

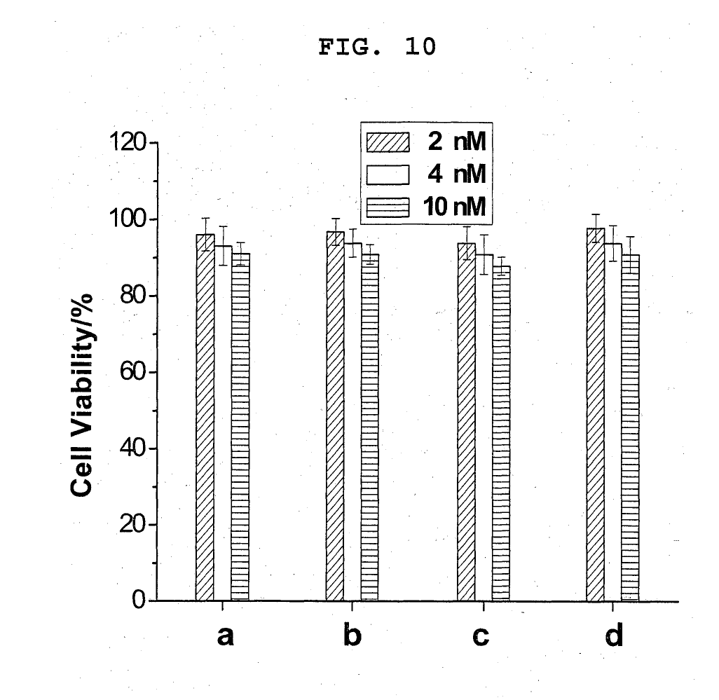

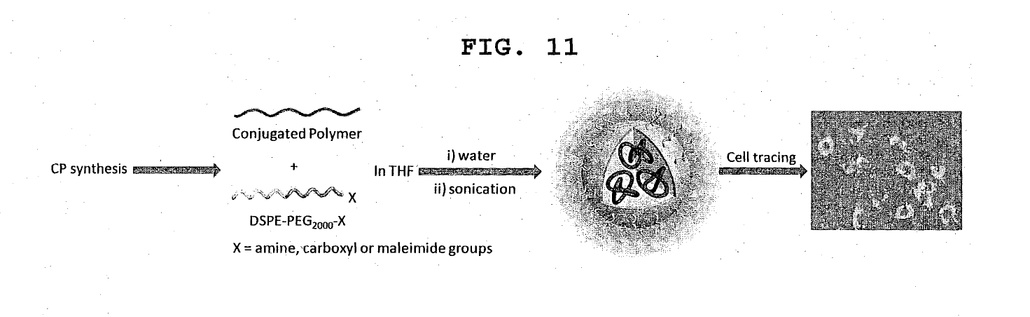

The cytotoxicity of Tat-conjugated FPdots was evaluated by MCF-7 cells using MTT method after incubation with said Tat-conjugated FPdots in culture medium at concentrations of 2, 4, 10 nM for 48 h, respectively. The low cytotoxicity of Tat modified and original FPdots were witnessed by the over 85% cell viability within the tested period (FIG. 9). The results indicated that FPdots are promising fluorescence probes for long term clinical applications. The photostability of these FPdots were also evaluated by measuring the fluorescence change of Tat-conjugated FPdot-treated MCF-7 cells under continuous laser scanning with a 488 nm laser. The fluorescence changes for Qtracker™ 585, Alexa Fluor 488 and FITC were also measured under the same conditions for comparison. As shown in FIG. 10, FPdots displayed a comparable fluorescence loss (˜9%) to Qtracker™ 585 (˜6%), which is obviously less as compared to Alexa Fluor 488 (˜14%) and FITC (˜60%) under the same conditions.

### APPLICATIONS

The disclosed FPdots may be of uniform size and low cytotoxicity which makes them useful for bioimaging purposes.

The disclosed FPdots may be of comparable size, similar or superior photostability and similar or superior labelling efficiency and possess low cytotoxicity when compared with inorganic quantum dots and may therefore be superior alternatives in bioimaging applications.

The size of the disclosed FPdots may be customizable. As the electronic characteristics of FPdots may be affected by its size, the disclosed FPdots may be customized to provide a range of electronic characteristics that suit different needs.

The size of the disclosed FPdots advantageously may not change after time, e.g. after storage, thereby displaying good stability.

The disclosed FPdots may comprise functional group(s) which may be customizable, allowing for a wide variety of biomolecules to be conjugated to the FPdot allowing for a greater variety of bioimaging applications.

The disclosed FPdots may exhibit similar or superior performance in molar extinction coefficient, fluorescence quantum yield and long-term stability when compared with quantum dots or conventional conjugated polymer-based nanoparticles which is of importance in long-term cell tracing applications.

The disclosed FPdots may display superior living cell tracing ability up to about 12 generations.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A fluorescent polymer dot comprising one or more fluorescent conjugated polymers, forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties.

2. The fluorescent polymer dot according to claim 1, wherein the one or more aliphatic chain moieties are saturated.

3. The fluorescent polymer according to claim 2, wherein the one or more saturated aliphatic chain moieties comprises 10 to 24 carbon atoms.

4. The fluorescent polymer dot according to any one of claims 2 to 3, wherein the one or more saturated aliphatic chain moieties are part of a lipid moiety.

5. The fluorescent polymer dot according to claim 4, wherein the lipid moiety is selected from the group consisting of 1,2-distearoyl-sn-glycero-3-phosphoethanolamino (DSPE) moiety, 1,2-Dimyristoyl-sn-glycero-3-phosphoethanolamine (DMPE) moiety, 1,2-Dilauroyl-sn-glycero-3-phosphoethanolamine (DLPE) moiety, and (1,2-dipalmitoyl-sn-glycero-3-phosphoethanolamine) (DPPE) moiety.

6. The fluorescent polymer dot according to any one of claims 1 to 5, wherein the hydrophilic end of the amphiphilic molecule comprises one or more polymers selected from the group consisting of polyoxyalkylene, polyalkylene glycol, and polycarboxyalkylene.

7. The fluorescent polymer dot according to any one of claims 1 to 6, wherein the amphiphilic molecule comprises one or more polymers selected from the group consisting of polyethylene glycol (PEG), polypropylene glycol, polybutylene glycol, polycarboxymethylene, polycarboxyethylene (or polyacrylic acid (PAA)), polycarboxypropylene, and polycarboxybutylene.

8. The fluorescent polymer dot according to any one of claims 1 to 7, wherein the hydrophilic end of the amphiphilic molecule is attached to one or more functional groups.

9. The fluorescent polymer dot according to claim 8, wherein the functional group is selected from the group consisting of alkoxy, amino, carboxy, imide, and biotin.

10. The fluorescent polymer dot according to any one of claims 1 to 9, wherein the amphiphilic molecule is selected from the group consisting of:
1,2-distearoyl-sn-glycero-3-phosphoethanolamine-N-[(polyethylene glycol)-2000] (DSPE-PEG);
1,2-distearoyl-sn-glycero-3-phosphoethanolamine-N-[methoxy(polyethylene glycol)-2000] (DSPE-PEG-OCH₃),
1,2-Distearoyl-sn-glycero-3-phosphoethanolamine-N-[amino(polyethylene glycol)-2000] (DSPE-PEG-NH₂);
1,2-distearoyl-sn-glycero-3-phosphoethanolamine-N-[carboxy(polyethylene glycol)-2000] (DSPE-PEG-COOH);
1,2-distearoyl-sn-glycero-3-phosphoethanolamine-N-[maleimide(polyethylene glycol)-2000] (DSPE-PEG-maleimide);
1,2-distearoyl-sn-glycero-3-phosphoethanolamine-N-[biotinyl(polyethylene glycol)-2000] (DSPE-PEG-biotin);
1,2-Dimyristoyl-sn-glycero-3-phosphoethanolamine-N-[(polyethylene glycol)-2000] (DMPE-PEG);
1,2-Dimyristoyl-sn-glycero-3-phosphoethanolamine-N-[methoxy(polyethylene glycol)-2000] (DMPE-PEG-OCH₃);
1,2-Dimyristoyl-sn-glycero-3-phosphoethanolamine-N-[amino(polyethylene glycol)-2000] (DMPE-PEG-NH₂);
1,2-Dimyristoyl-sn-glycero-3-phosphoethanolamine-N-[carboxy(polyethylene glycol)-2000] (DMPE-PEG-COOH);
1,2-Dimyristoyl-sn-glycero-3-phosphoethanolamine-N-[maleimide(polyethylene glycol)-2000] (DMPE-PEG-maleimide);
1,2-Dimyristoyl-sn-glycero-3-phosphoethanolamine-N-[biotinyl(polyethylene glycol)-2000] (DMPE-PEG-Biotin);
1,2-Dilauroyl-sn-glycero-3-phosphoethanolamine-N-[(polyethylene glycol)-2000] (DLPE-PEG);
1,2-Dilauroyl-sn-glycero-3-phosphoethanolamine-N-[methoxy(polyethylene glycol)-2000] (DLPE-PEG-OCH₃);
1,2-Dilauroyl-sn-glycero-3-phosphoethanolamine-N-[amino(polyethylene glycol)-2000] (DLPE-PEG-NH₂);
1,2-Dilauroyl-sn-glycero-3-phosphoethanolamine-N-[carboxy(polyethylene glycol)-2000] (DLPE-PEG-COOH);
1,2-Dilauroyl-sn-glycero-3-phosphoethanolamine-N-[maleimide(polyethylene glycol)-2000] (DLPE-PEG-maleimide)-;
1,2-Dilauroyl-sn-glycero-3-phosphoethanolamine-N-[biotinyl(polyethylene glycol)-2000] (DLPE-PEG-Biotin);
1,2-dipalmitoyl-sn-glycero-3-phosphoethanolamine-N-[(polyethylene glycol)-2000] (DPPE-PEG);
1,2-dipalmitoyl-sn-glycero-3-phosphoethanolamine-N-[methoxy(polyethylene glycol)-2000] (DPPE-PEG-OCH₃);
1,2-dipalmitoyl-sn-glycero-3-phosphoethanolamine-N-[amino(polyethylene glycol)-2000] (DPPE-PEG-NH₂);
1,2-dipalmitoyl-sn-glycero-3-phosphoethanolamine-N-[carboxy(polyethylene glycol)-2000] (DPPE-PEG-COOH);
1,2-dipalmitoyl-sn-glycero-3-phosphoethanolamine-N-[maleimide(polyethylene glycol)-2000] (DPPE-PEG-maleimide); and
1,2-dipalmitoyl-sn-glycero-3-phosphoethanolamine-N-[biotinyl(polyethylene glycol)-2000] (DPPE-PEG-Biotin);
1,2-distearoyl-sn-glycero-3-phosphoethanolamine-N-[(polyacrylic acid)] (DSPE-PAA);
1,2-distearoyl-sn-glycero-3-phosphoethanolamine-N-[methoxy(polyacrylic acid)] (DSPE-PAA-OCH₃),
1,2-Distearoyl-sn-glycero-3-phosphoethanolamine-N-[amino(polyacrylic acid)] (DSPE-PAA-NH₂);
1,2-distearoyl-sn-glycero-3-phosphoethanolamine-N-[carboxy(polyacrylic acid)] (DSPE-PAA-COOH);
1,2-distearoyl-sn-glycero-3-phosphoethanolamine-N-[maleimide(polyacrylic acid)] (DSPE-PAA-maleimide);
1,2-distearoyl-sn-glycero-3-phosphoethanolamine-N-[biotinyl(polyacrylic acid)] (DSPE-PAA-biotin);
1,2-Dimyristoyl-sn-glycero-3-phosphoethanolamine-N-[(polyacrylic acid)] (DMPE-PAA);
1,2-Dimyristoyl-sn-glycero-3-phosphoethanolamine-N-[methoxy(polyacrylic acid)] (DMPE-PAA-OCH₃);
1,2-Dimyristoyl-sn-glycero-3-phosphoethanolamine-N-[amino(polyacrylic acid)] (DMPE-PAA-NH₂);
1,2-Dimyristoyl-sn-glycero-3-phosphoethanolamine-N-[carboxy(polyacrylic acid)] (DMPE-PAA-COOH);
1,2-Dimyristoyl-sn-glycero-3-phosphoethanolamine-N-[maleimide(polyacrylic acid)] (DMPE-PAA-maleimide);
1,2-Dimyristoyl-sn-glycero-3-phosphoethanolamine-N-[biotinyl(polyacrylic acid)] (DMPE-PAA-Biotin);
1,2-Dilauroyl-sn-glycero-3-phosphoethanolamine-N-[(polyacrylic acid)] (DLPE-PAA);
1,2-Dilauroyl-sn-glycero-3-phosphoethanolamine-N-[methoxy(polyacrylic acid)] (DLPE-PAA-OCH₃);
1,2-Dilauroyl-sn-glycero-3-phosphoethanolamine-N-[amino(polyacrylic acid)] (DLPE-PAA-NH₂);
1,2-Dilauroyl-sn-glycero-3-phosphoethanolamine-N-[carboxy(polyacrylic acid)] (DLPE-PAA-COOH);
1,2-Dilauroyl-sn-glycero-3-phosphoethanolamine-N-[maleimide(polyacrylic acid)] (DLPE-PAA-maleimide);
1,2-Dilauroyl-sn-glycero-3-phosphoethanolamine-N-[biotinyl(polyacrylic acid)] (DLPE-PAA-Biotin);
1,2-dipalmitoyl-sn-glycero-3-phosphoethanolamine-N-[(polyacrylic acid)] (DPPE-PAA);
1,2-dipalmitoyl-sn-glycero-3-phosphoethanolamine-N-[methoxy(polyacrylic acid)] (DPPE-PAA-OCH₃);
1,2-dipalmitoyl-sn-glycero-3-phosphoethanolamine-N-[amino(polyacrylic acid)] (DPPE-PAA-NH₂);
1,2-dipalmitoyl-sn-glycero-3-phosphoethanolamine-N-[carboxy(polyacrylic acid)] (DPPE-PAA-COOH);
1,2-dipalmitoyl-sn-glycero-3-phosphoethanolamine-N-[maleimide(polyacrylic acid)] (DPPE-PAA-maleimide); and
1,2-dipalmitoyl-sn-glycero-3-phosphoethanolamine-N-[biotinyl(polyacrylic acid)] (DPPE-PAA-Biotin).

11. The fluorescent polymer dot according to any one of claims 1 to 10, wherein the fluorescent conjugated polymer comprises repeating units of optionally substituted cyclic group linked to O-containing heteroaryl group.

12. The fluorescent polymer dot according to claim 11, wherein the optionally substituted cyclic group is an optionally substituted monocyclic or polycyclic group.

13. The fluorescent polymer dot according to claim 12, wherein the optionally substituted monocyclic or polycyclic group is aromatic.

14. The fluorescent polymer dot according to any one of claims 11 to 13, wherein the O-containing heteroaryl group is monocyclic or polycyclic.

15. The fluorescent polymer dot according to any one of claims 11 to 14, wherein the O-containing heteroaryl group is selected from the group consisting of diazole, benzodiazole, oxazole, benzooxazole, pyran, furan, and benzofuran.

16. The fluorescent polymer dot according to any one of claims 11 to 15, wherein the fluorescent conjugated polymer is poly(9,9-dihexylfluorene-alt-2,1,3-benzoxadiazole) (PFBD).

17. The fluorescent polymer dot according to any one of claims 1 to 16, wherein a biological molecule is conjugated to the surface of said dot.

18. The fluorescent polymer dot according to any one of claims 8 to 17, wherein a biological molecule is conjugated to the surface of the dot via the functional group.

19. The fluorescent polymer dot according to any one of claim 17 or 18, wherein the biological molecule is selected from the group consisting of amino acids, peptides, polypeptides, nucleic acids, carbohydrates, lipids, fatty acids, antibodies, aptamers, and proteins.

20. The fluorescent polymer dot according to any one of claims 17 to 19 wherein the biological molecule is a cell penetrating peptide.

21. The fluorescent polymer dot according to claim 20, wherein the cell penetrating peptide is a Tat peptide.

22. The fluorescent polymer dot according to any one of claims 1 to 21, wherein the weight ratio of fluorescent conjugated polymer to amphiphilic molecule is in the range of 25% to 75%.

23. The fluorescent polymer dot according to any one of claims 1 to 22, wherein the size of said dot is in the range of 30 nm to 50 nm.

24. A method for preparing a fluorescent polymer dot, the method comprising the steps of:
a) preparing a mixture of fluorescent conjugated polymer and amphiphilic molecule in an aprotic solvent; and
b) adding said mixture to a protic solvent, to form the polymer dot comprising one or more fluorescent conjugated polymers forming a hydrophobic core and one or more amphiphilic molecules, each amphiphilic molecule comprising a hydrophobic end embedded in the core and a hydrophilic end that forms a hydrophilic shell surrounding the core, wherein said hydrophobic end comprises one or more aliphatic chain moieties.

25. The method according to claim 24, wherein the aprotic solvent is tetrahydrofuran or acetone.

26. The method according to any one of claims 24 to 25, wherein the protic solvent is water.

27. The method according to any one of claims 24 to 26, further comprising the step of controlling the size of the fluorescent polymer dot produced, the step comprising modifying the initial concentration of fluorescent conjugated polymer in aprotic solvent.

28. The method according to claim 27, wherein the initial concentration of fluorescent conjugated polymer in aprotic solvent is in the range of 0.05 mg/mL to 1.50 mg/mL.

29. The method according to any one of claims 24 to 28, wherein the amphiphilic molecule is attached to one or more functional groups.

30. The method according to any one of claims 24 to 29, further comprising the step of conjugating a biological molecule to the surface of the fluorescent polymer dot.

31. The method according to claim 30, wherein the biological molecule is conjugated to the fluorescent polymer dot via the one or more functional groups.

32. A method of detecting a target molecule in a biological sample, the method comprising contacting a biological sample with a fluorescent polymer dot as claimed in any one of claims 1 to 23.
